(19) United States
(12) Patent Application Publication
(10) Pub. No.: US 2007/0078364 A1
(43) Pub. Date: Apr. 5, 2007

(54) Wound cover/stabilizer

(76) Inventor: Linda Byrl Davis Belcher, Hurley, VA (US)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 11/291,904

(22) Filed: Dec. 2, 2005

Related U.S. Application Data
(63) Continuation-in-part of application No. 11/205,814, filed on Aug. 17, 2005.

(51) Int. Cl. A61F 15/00; A61F 13/00

(52) U.S. Cl. 602/42; 602/78

(57) ABSTRACT

The invention provides a stabilizing cover to wear over an IV site or any type of wound that requires a protective pad and needs to be accessed or observed often. The device's opening/closure allows doctors, nurses, and other care-givers to check the wound site without the discomfort and inconvenience of removing adhesive tape that is typically used to secure a bandage to the wound site. Using the present invention, IV sites may be checked or changed without pulling up or down and possibly pulling the IV out. The cover also can secure gauze or other wound coverings over the wound site without use of adhesives on the patient's skin. Various sizes of snag-free fasteners, in accordance with the present invention, are used to simplify and expedite the process of checking the site. The stabilizing cover is made of fabric that is flexible, comfortable and disposable.

### CROSS REFERENCE TO RELATED APPLICATIONS

This application is a continuation-in-part of U.S. patent application Ser. No. 11/205,814, filed on Aug. 15, 2005, which is incorporated herein by reference in its entirety.

### STATEMENT REGARDING SPONSORED RESEARCH OR DEVELOPMENT

Not Applicable

### REFERENCE TO SEQUENCE LISTING

Not Applicable

### BACKGROUND OF THE INVENTION

1. Field of the Invention

The present invention relates generally to the field of protective devices for body parts with dermal wounds in both human and non-human patients. More particularly the present invention relates to single-piece devices which protect sites for IV needles, catheters, or small wounds from contamination while partially stabilizing any needles and/or connecting tubes at the site.

2. Description of Related Art

Dermal wounds treated in a hospital or clinical setting often require frequent observation by medical personnel. Such wounds may be the result of an accident to a patient or may be required during medical treatment, such as in intravenous needle or catheter insertion. Except for during periods of observation or treatment by medical personnel, it is typically preferable—for a variety of reasons—to cover the dermal wound site. The covering, of course, must then be removed to access or observe the wound site.

Intravenous needles allow a medical-care-provider to inject liquids into a patient's vein. A needle is inserted into the patient's vein, typically by a nurse or other medical personnel. A delivery tube connected to the needle is typically secured to the anatomy of the patient with adhesive tape in order to try and relieve any stress on the injected region and to try to prevent or at least reduce the risk of the needle pulling out from the vein if the tube is inadvertently pulled. In this normal arrangement, the needle is exposed to the atmosphere, except for the portion inserted into the vein, and thus dirt, fluids or contaminants could come in contact with the needle and tube at the site of insertion. This typical arrangement may also cause discomfort associated with removal of the needle when removing adhesive tape from the patient's skin. Furthermore, some patients may have damaged skin, skin allergies or sensitive skin which would make use of any adhesive tape uncomfortable for the patient.

Previous attempts have been made to design covers for IV needles and the associated tube as well for holding and protecting the IV needles when inserted into a patient. Such designs are typically limited to insertion of the needle into a vein in a patient's arm or back of the hand adjacent the wrist. Most are not suitable for use when the needle is inserted into the vicinity of a joint, such as a metacarpophalangeal joint, or other body part that does not maintain a stationary surface, such as the abdomen or thigh. The difficulty with these areas is that the extent of flexing prevents effective stabilization throughout the entire range of motion of the joint or other body part. In contrast, the present invention discloses a design embodiment which is suitable for IV needle insertions or wound sites in these areas.

Several designs for covers have also been made that do not completely prevent visibility of the IV or catheter injection site. While continual visibility of a wound or injection site may be beneficial to the care-provider in some cases, the ability to completely prevent visibility of such sites is often desirable in cases where a patient, such as a child for example, may be emotionally disturbed by observing the wound or injection site.

Other previous cover designs provide a removable panel or widow to access to a wound site, while the remainder of the cover remains secured to the patient. Additional clips or exposed fasteners may also be included in these designs. Such designs add cost and complexity to the cover. Furthermore, the additional closures and complexity of these designs gives these devices a more intimidating appearance that may be emotionally disruptive some patients.

Still other cover designs have been made that include multiple layers of material, including a gauze layer affixed to the interior of the cover to directly cover the wound site. These designs also add additional cost and complexity. While such designs provide the convenience of using a single component to cover the wound site, the orientation of the cover when applied to the patient is limited to that which aligns the gauze with the wound site. Thus, these designs are limited in their effective range of use to only those applications where the gauze in the cover can be properly aligned with the wound.

The need exists for a stabilizing wound cover for the protection of small wounds and the indwelling catheter access sites that simultaneous provides absorbency of moisture, obstructs visibility of the site when secured, provides easy access to the skin or cannula exit point, provides mechanical stabilization of an indwelling catheter or sterile pad, and allows for easy removal without discomfort to the patient. There further exists a need for a wound cover that is simple, cost effective, an not intimidating to the patient or user.

### SUMMARY OF THE INVENTION

To address the aforementioned needs, the invention provides a stabilizing cover to wear over an IV site or any type of wound that requires a protective pad and needs to be accessed or observed often. The device's opening/closure allows doctors, nurses, and other care-givers to check the wound site without patient discomfort and the inconvenience of removing adhesive tape that is typically used to secure a bandage to the wound site. Using the present invention, IV sites may be checked or changed without pulling up or down and possibly pulling the IV out. The cover also can secure gauze or other wound coverings over the wound site without use of adhesives on the patient's skin. Various sizes of snag-free fasteners, in accordance with the present invention, are used to simplify and expedite the process of checking the site. The stabilizing cover is made of fabric that is flexible, comfortable and disposable.

The device prevents visibility of the wound site while having a soft and uncomplicated appearance, which makes it especially well-suited for use with infants or small children who may be uncomfortable with the sight of an IV site or complicated site cover. In one embodiment the fabric may be substantially liquid impermeable to prevent fluid leakage from the covered area. In an alternate embodiment the material may be absorptive to hold leakage away from the wound site. In another aspect of the invention, the cover is preferably sterile.

In one embodiment of the invention a disposable stabilizing cover for a wound site is provided, the cover being free of any protruding fasteners (protrusion-free), covering the wound and securing any associated padding. The cover includes an elastic panel of breathable material that has an upper edge, a lower edge, a first side edge, and a second side edge. The cover further includes a reclosable fastener system for securing the first side edge to the second side edge so as to form a sleeve, wherein the secured panel encircles a body part to cover the wound site with sufficient pressure to secure the associated padding in place over the wound without excessive pressure on the wound site and without use of any adhesive.

Another embodiment of the invention provides a protrusion-free, disposable, stabilizing cover for an intravenous needle insertion site that covers the insertion point and secures any associated padding and tubing at the insertion site. The cover includes a single, substantially-rectangular, elastic panel of breathable material, the panel including an upper edge, a lower edge, a first side edge, and a second side edge. The cover also includes a reclosable fastener system for securing the first side edge to the second side edge so as to form a sleeve, wherein the secured panel encircles a body part to cover the needle insertion site with sufficient pressure to stabilize the needle and associated tubing without exerting excessive pressure on the wound site.

### BRIEF DESCRIPTION OF THE DRAWINGS

The accompanying drawings are included to provide further understanding of the invention and are incorporated in and constitute a part of this specification. The accompanying drawings illustrate embodiments of the invention and together with the description serve to explain the principles of the invention. In the figures:

FIG. 1 is a top plan view of a stabilizing cover applied to a patient's hand in accordance with a typical embodiment of the present invention;

FIG. 2 is a top plan view of a stabilizing cover applied to a patient's forearm in accordance with a typical embodiment of the present invention;

FIG. 3 is a side plan view of a stabilizing cover applied to a patient's calf in accordance with a typical embodiment of the present invention;

FIG. 4 is a side plan view of a stabilizing cover applied to a patient's thigh in accordance with a typical embodiment of the present invention;

FIG. 5 is a side plan view of a stabilizing cover applied to a patient's foot in accordance with a typical embodiment of the present invention;

FIG. 6 is a side plan view of a stabilizing cover applied to a patient's ankle in accordance with a typical embodiment of the present invention;

FIG. 7 is a side plan view of two separate stabilizing covers applied to a patient's head and neck, respectively, in accordance with a typical embodiment of the present invention;

FIG. 8 is a side plan view of a stabilizing cover applied to a patient's abdomen in accordance with a typical embodiment of the present invention; and

FIG. 9 is a schematic of a typical wound site just prior to application of the stabilizing cover of the present invention.

### DETAILED DESCRIPTION OF THE INVENTION

Reference will now be made in detail to the preferred embodiments of the present invention, examples of which are illustrated in the accompanying drawings.

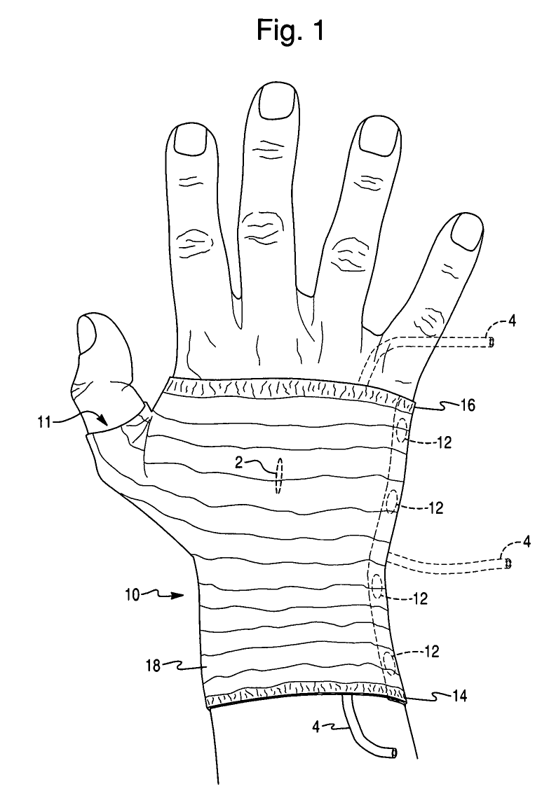

FIG. 1 shows a wound cover 10, as applied to a patient's hand in one embodiment of the invention. The wound cover 10 covers an IV site 2 or other wound site that is located on the back of a patent's hand and that needs to be accessed or observed often. The cover includes a elastic panel 18 of material that is wrapped around the patent's hand to cover the wound site. The material for elastic panel 18 may be breathable, absorptive, and latex-free. The panel 18 is also, preferably, sterile prior to application.

The cover 10 includes a reclosable fastener system 12. The fastener system 12 may include hook-and-loop fasteners, such as Velcro® brand fasteners, secured to the opposite ends and opposites sides of the elastic panel 18. The fasteners may, for example, be sewn or adhesively bonded to panel 18. When in any secured position, the hook-and-loop fasteners of fastener system 12 should be covered by part of elastic panel 18, so as to avoid any protrusions and prevent snagging the cover on other objects or materials. Alternatively, the fastener system 12 may use an adhesive applied to the underside of one end of elastic panel 18 to be secured against the top side of the other end of elastic panel 18. The use of snaps or buttons are also contemplated for use in fastener system 12. Using the reclosable fastener system 12, the cover 10 can be quickly and easily applied to minimize the amount of time the wound site 2 is visible and/or exposed to potential contaminants.

While FIG. 1 shows use of four fastening points, fewer or more fastening points may be used for fastener system 12, including a continuous fastening strip extending from the lower edge 14 to the upper edge 16. The fastening system 12 shown in FIG. 1 is oriented vertically. However, it is also contemplated that the fasteners could be oriented horizontally with a fastener strip at least along a portion of the lower edge 14 and the upper edge 16. This arrangement of fastening system 12 would allow for a greater range of adjustability for the wound cover 10.

In the embodiment shown in FIG. 1, cover 10 includes an opening 11 for accommodating the patient's thumb. The remaining fingers of the patient's hand extend though the opening formed at upper edge 16 when the cover 10 is secured.

Elastic panel 18 may be composed of an elastomeric, cloth-like, nonwoven fibrous material, such as an elastomeric stretch-bonded laminate (SBL) web or an elastomeric meltblown web. These materials have been widely used in other applications, such as elastic sections of diapers and other disposable undergarments. In a particular aspect of the present invention, elastic panel 18 is composed of an elastomeric, nonwoven fibrous web which is substantially air-permeable and vapor-permeable. In a particular embodiment of the invention, the elastic panel 18 has a porosity of between about 3 cfm/sq. ft. and about 10 cfm/sq ft. The fibrous, nonwoven, elastomeric material employed in the elastic panel 18 of the present invention provides a relatively soft surface which is less likely to irritate the skin of the wearer, while the permeability allows for healing of the wound site.

In another aspect of the invention, elastic panel 18 can be composed of an elastomeric, stretchable composite web comprising individual, discrete strips of elastomeric material secured to one or more nonwoven fibrous layers. Such a composite web may, for example, comprise an elastomeric meltblown material arranged in a selected pattern of strips and suitably sandwiched and attached between two layers of nonwoven, spunbond, fibrous material. The composite web may alternatively comprise a selected pattern of individual elastomer strips operably secured to a nonwoven fibrous layer or between two nonwoven layers. The elastomer strips may, for example, be composed of a thermoplastic, melt extrudable material. Examples of suitable elastomer materials include polyether-polyamide block copolymers, polyurethanes, synthetic linear A-B-A and A-B block copolymers, chlorinated rubber/EVA (ethylene-vinyl acetate) blends, EPDM (ethylene-propylene diene monomer) rubbers, EPM (ethylene-propylene monomer) rubbers, blends of EPDM/EPM/EVA, and the like. The resultant panel has a substantially continuous gather of nonwoven material along the entire length of elastic panel 18.

In a further aspect of the invention elastic panel 18 may comprise a woven fabric. For example, similar to the methods described above for nonwoven material, an elastic or expansible layer is expanded or stretched to some extent, the woven non-stretched layer is placed on the stretched elastic layer, these layers are bonded together to form a laminate, and the laminate is relaxed so that the elastic layer makes a gathering of the remaining woven layer.

Elastic panel 18 should be sufficiently flexible to easily conform to various widths of the patient's hand. Thus, the panel 18 may generally have a simple rectangular shape before being wrapped around the patient and secured. Preferably, elastic panel 18 should be able to expand from an unstretched position to at least double the unstretched length. The cover 10 can conform to a wide range of dimensions without overlapping bandage layers or encircling the wound site multiple times. In an alternate embodiment, the panel 18 may be formed with a gradually increasing width dimension extending from a slightly narrower width extending along lower edge 14 to a larger width along upper edge 16. Using any of the above described embodiments, cover 10 may also be used to secure a gauze pad or other pad, in place over the wound site.

If the wound site 2, includes an IV needle (not shown) and tubing 4, the tubing 4 is stabilized by the cover 10. The material of elastic panel 18 provides a snug fit to limit movement of the tubing 4, while providing sufficient flexibility to prevent uncomfortable pressure on the IV wound site 2. As an example, the contracting stress of the elastic panel 18 in the areas adjacent to the wound are preferably in the range from about 30 g/25 mm width to about 300 g/25 mm width at a 120% to 200% stretch. With the cover 10 applied to the patient, the tubing 4 may pass under the bandage through openings formed at lower edge 14 and upper edge 16 when the cover 10 is secured. As shown in FIG. 1, the tubing 4 may also exit the cover 10 by passing between fastening points of fastening system 12. In an alternative embodiment, the portions of panel 18 near the upper edge 16 and the lower edge 14 may contain additional elastic stitching or be more tightly gathered to form a more secure cuff along the primary openings of the cover 10 while maintaining a less firm pressure on the actual wound site.

The expansion or stretch of elastic panel 18 is preferably limited to substantially one direction, specifically from side to side. Limiting the direction of the stretch of panel 18 allows for improved stabilizing of an underlying wound cover or IV tubing. Alternatively, the elastic panel 18 could have elastic stresses in multiple directions. In this alternative embodiment, the degree of expansion may vary so that side-to-side expansion could be significantly greater than in other directions.

With the exception of fastener system 12, the cover 10 is completely free of any tube tie-downs, clamps, holders, tape layers, brackets or other protrusions. When secured, the gathered material of panel 18, while not smooth, forms a cuff with a surface that is sufficiently even to avoid inadvertently catching or snagging. The cover 10 is flexible and lightweight, to minimize any discomfort, while completely preventing a view of the covered wound and/or any associated needles and tubing. Symbols or pictures may also be printed on or otherwise affixed to panel 18 to create a more appealing appearance. Alternatively, patches may be provided that can be affixed to the cover after application to ensure visibility and proper orientation when viewed by the patient. The cover 10 does not require additional straps or casing to support panel 18, thus simplifying the design and reducing manufacturing costs.

The cover 10 uses a single-layered panel 18 that does not include gauze or other materials on the inner surface of the panel. Instead, if required, gauze or padding is placed directly on the wound prior to applying the cover 10, and the gauze or padding is then secured by the cover 10. This arrangement allows for a simpler cover design and ensures that the mating surfaces of the secured panel can be located at any orientation relative to the wound location without concern for the location of the underlying pad. Similarly, if IV tubing is present in the wound site, the cover can be rotated to accommodate the ideal exit location for the tubing without concern for aligning any gauze or additional padding. Because the cover 10 does not require orientation of the gauze when applied to the patient, the cover 10 can be used for a greater variety of applications than other cover designs that have gauze secured on a portion of the inside of those covers.

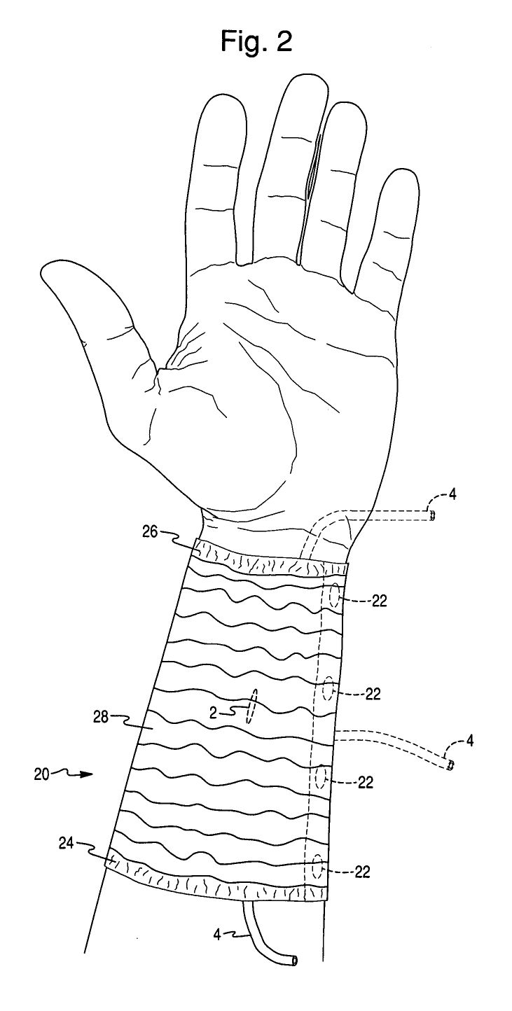

FIG. 2 shows a wound cover 20, as applied to a patient's arm in one embodiment of the invention. The wound cover 20 covers an IV site 2 or other wound site that is located on the patent's arm, above the wrist, and that needs to be accessed or observed often. The cover 20 includes elastic panel 28, which has a lower edge 24 and an upper edge 26, and reclosable fastening system 22. The material and structure of the wound cover 20 are generally the same at that described above with respect to FIG. 1, except that no additional opening for the patient's thumb is required. Referring specifically to FIG. 2, the material for elastic panel 28 should be sufficiently flexible to easily conform to various widths of the patient's arm. Thus, the panel 28 will generally have a simple rectangular shape before being wrapped around the patient and secured. In an alternate embodiment, the panel 28 may be formed with a gradually varying width dimension changing from a slightly larger width extending along lower edge 24 to a narrower width along the upper edge 26. As discussed above with respect to the panel 18 of FIG. 1, the panel 28 of FIG. 2 may have a uniform elasticity or, alternatively, may be more tightly gathered along the lower edge 24 and/or the upper edge 26. If IV tubes 4 are present at the wound site 2, the tubes 4 may exit between fasteners 22 or at either the lower edge 24 or upper edge 26.

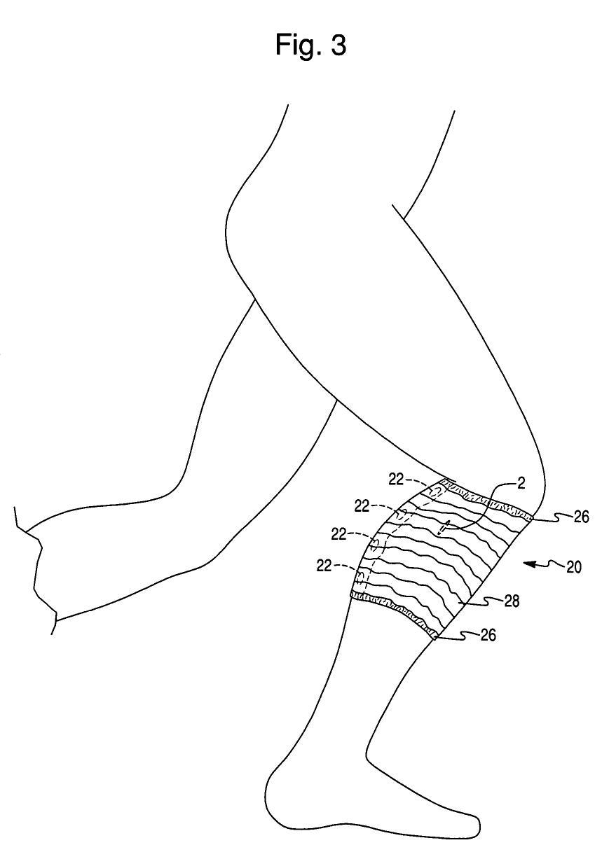

FIG. 3 shows the wound cover 20, as applied to a patient's leg in another embodiment of the invention. The wound cover 20 covers an IV site 2 or other wound that is located on the patent's leg, and more particularly on the patient's calf, that needs to be accessed or observed often. The material and structure of the wound cover 20 are generally the same at that described above with respect to the previous figures. Referring specifically to FIG. 3, the material for the elastic panel 28 should be sufficiently flexible to easily conform to various widths of the patient's leg. Thus, the panel 28 will generally have a simple rectangular shape before being wrapped around the patient and secured. In an alternate embodiment, the panel 28 may be formed with a gradually varying width dimension changing from a slightly smaller width extending along lower edge 24 to a larger width along the upper edge 26.

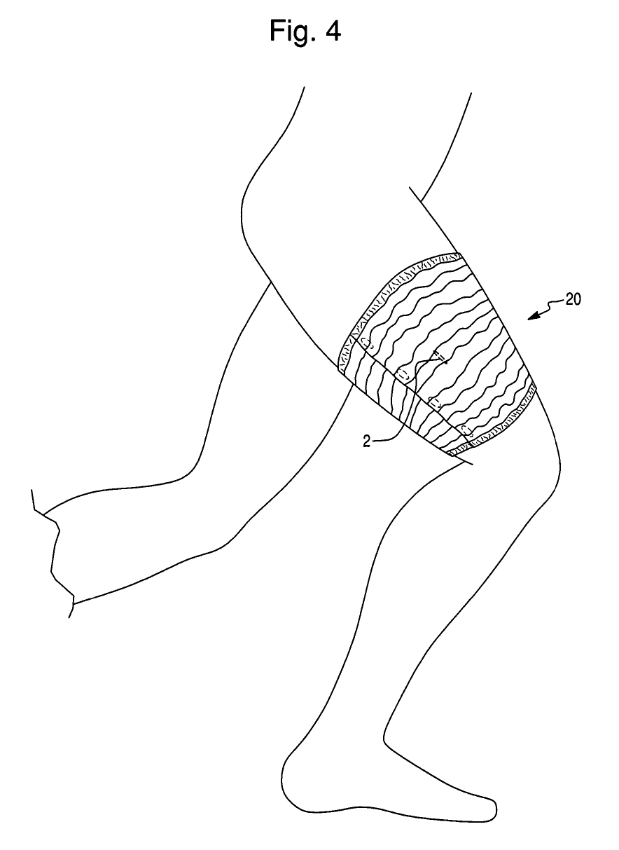

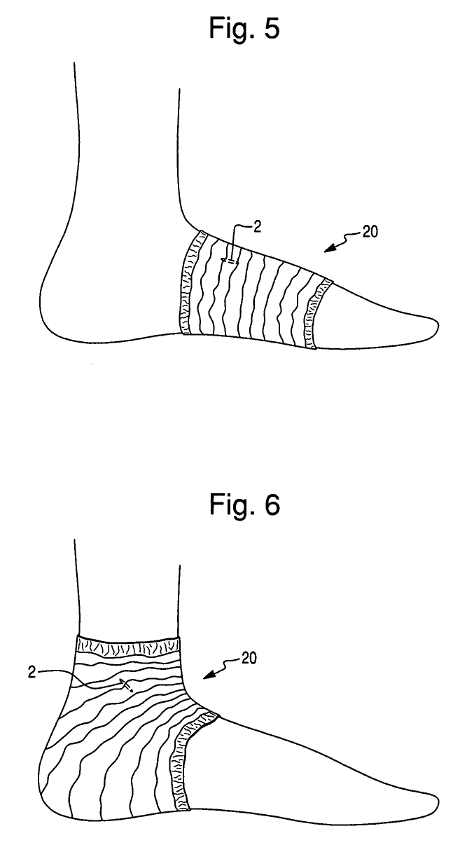

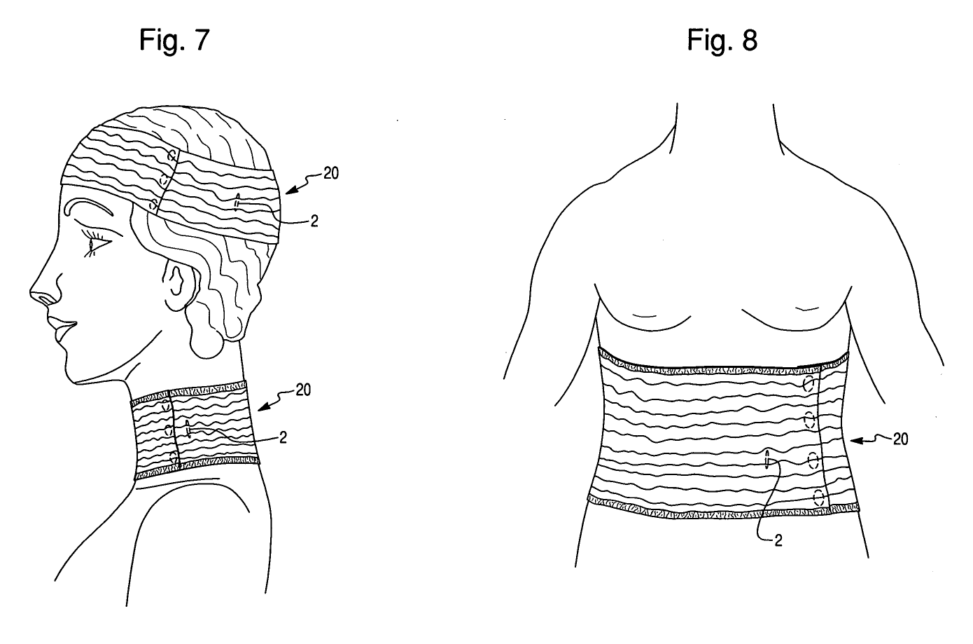

FIGS. 4 through 8 show the wound cover 20 applied to other various locations on a patient. Specifically, FIG. 4 shows the wound cover 20 as applied to the wound site 2 on a patient's thigh. FIG. 5 shows the wound cover 20 applied to the wound site 2 on top of a foot. FIG. 6 shows the wound cover 20 applied to the wound site 2 near a patient's ankle. FIG. 7 shows two separate wound covers 20 applied to the wound sites 2 on a patient's head and neck, respectively. FIG. 8 shows the wound cover 20 applied to the wound site 2 on a patient's abdomen.

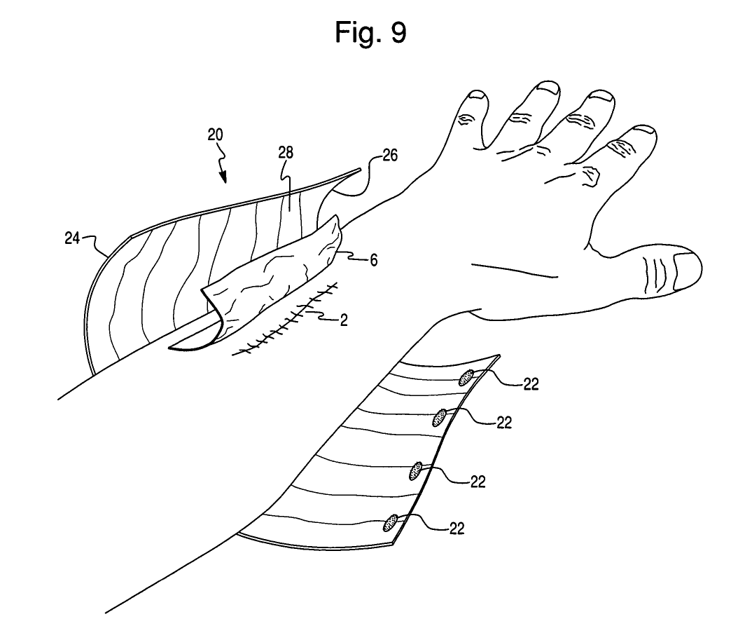

FIG. 9 shows the surgical or wound site 2 being covered with sterile gauze pad 6. The pad 6 is covered with stabilizing cover 20 to hold the gauze 6 in place. As described in greater detail above with respect to cover 10 of FIG. 1, the cover 20 uses a single-layered panel 28 and does not contain a separate gauze layer. The gauze pad 6 is placed directly on the wound site 2 prior to applying the cover 20. The gauze is then secured at the wound site when the cover 20 is secured with the reclosable fastening system 22. Because the gauze 6 is not connected to the cover 20, there is no need to orient the cover to align the gauze 6 with the wound site 2. In the embodiment shown in FIG. 9, the panel 28 does not include additional elastic gathering at the lower edge 25 and the upper edge 26, although such features are contemplated.

While exemplary embodiments of the invention have been shown and described herein, it will be obvious to those skilled in the art that such embodiments are provided by way of example only. Numerous insubstantial variations, changes, and substitutions will now be apparent to those skilled in the art without departing from the scope of the invention disclosed herein by the Applicants. Accordingly, it is intended that the invention be limited only by the spirit and scope of the claims, as they will be allowed.

1. A protrusion-free, disposable, stabilizing cover for a wound site that covers the wound and secures any associated padding, comprising:
an single continuous elastic panel of breathable material, the panel including an upper edge, a lower edge, a first side edge, and a second side edge;
a reclosable fastener system for securing the first side edge to the second side edge so as to form a sleeve, wherein the secured panel encircles a body part to cover the wound site with sufficient pressure to secure the associated padding in place over the wound without excessive pressure on the wound site and without use of any adhesive.

2. The stabilizing cover according to claim 1, wherein the reclosable fastener system is a mating strip combination of hook-and-loop fasteners secured along the first side edge and the second side edge.

3. The stabilizing cover according to claim 2, wherein the elastic panel comprises an elastomeric, cloth-like, nonwoven fibrous material.

4. The stabilizing cover according to claim 3, wherein a substantially continuous gather of the cloth-like, nonwoven fibrous material is formed along the entire length of the cover.

5. The stabilizing cover according to claim 4, wherein contracting stresses of the elastic panel are in the range from about 30 g/25 mm width to about 300 g/25 mm width at a 120% to 200% stretch.

6. The stabilizing cover according to claim 1, wherein the areas of the panel near the upper edge and the lower edge are more tightly gathered than the remaining area of the panel.

7. The stabilizing cover according to claim 1, wherein the elastic panel is absorptive.

8. The stabilizing cover according to claim 1, wherein the elastic panel is substantially liquid impermeable.

9. The stabilizing cover according to claim 1, wherein the elastic panel expands only along the direction between the first side edge and the second side edge.

10. A protrusion-free, disposable, stabilizing cover for an intravenous needle insertion site that covers the insertion point and secures any associated padding and tubing at the insertion site, said cover comprising:
a single, continuous, elastic panel of breathable material, the panel including an upper edge, a lower edge, a first side edge, and a second side edge;
a reclosable fastener system for securing the first side edge to the second side edge so as to form a sleeve, wherein the secured panel encircles a body part to cover the needle insertion site with sufficient pressure to stabilize the needle and associated tubing without causing excessive pressure on the wound site.

11. The stabilizing cover according to claim 10, wherein the reclosable fastener system is a mating strip combination of hook-and-loop fasteners secured along the first side edge and the second side edge.

12. The stabilizing cover according to claim 11, wherein the elastic panel comprises an elastomeric, cloth-like, nonwoven fibrous material.

13. The stabilizing cover according to claim 12, wherein a substantially continuous gather of the cloth-like, nonwoven fibrous material is formed along the entire length of the cover.

14. The stabilizing cover according to claim 13, wherein contracting stresses of the elastic panel are in the range from about 30 g/25 mm width to about 300 g/25 mm width at a 120% to 200% stretch.

15. The stabilizing cover according to claim 10, wherein the areas of the panel near the upper edge and the lower edge are more tightly gathered than the remaining area of the panel.

16. The stabilizing cover according to claim 10, wherein the elastic panel is absorptive.

17. The stabilizing cover according to claim 10, wherein the elastic panel is substantially liquid impermeable.

18. The stabilizing cover according to claim 10, wherein the elastic panel expands only along the direction between the first side edge and the second side edge.
